(19) United States
(12) Patent Application Publication
(10) Pub. No.: US 2020/0016064 A1
(43) Pub. Date: Jan. 16, 2020

(54) COLOURANT-INCORPORATED SILK FOR VALUE-ADDED COSMETICS

(71) Applicant: Agency for Science, Technology and Research, Singapore (SG)

(72) Inventors: Yin Win Khin, Singapore (SG); Choon Peng Teng, Singapore (SG); Michelle Dela Cruz Regulacio, Singapore (SG); Si Yin Tee, Singapore (SG); Leng Duei Koh, Singapore (SG); Michelle Low, Singapore (SG); Xian Jun Loh, Singapore (SG); Ming Yong Han, Singapore (SG)

(21) Appl. No.: 16/469,118

(22) PCT Filed: Dec. 20, 2017
(86) PCT No.: PCT/SG2017/050629

§ 371 Date: Jun. 12, 2019

(30) Foreign Application Priority Data
Dec. 20, 2016 (SG) 10201610640X

(51) Int. Cl. A61K 8/98; A61Q 1/02

(52) U.S. Cl. CPC A61K 8/987; A61Q 1/02; A61K 2800/805; A61K 2800/82; A61K 2800/43

(57) ABSTRACT

In the present invention, a method of producing a colourant-incorporated silk fibroin is provided. In a preferred embodiment, the method comprises the steps of: i) decrystallizing a degummed silk fibroin in the presence of an acidic solution or a metal salt solution (i.e. lithium bromide); ii) mixing one or more colourants with the decrystallized silk fibroin and iii) recrystallizing the decrystallized silk fibroin containing the colourants such that it forms a recrystallized region encapsulating the colourants, thereby obtaining the colourant-incorporated silk fibroin. In addition, a cosmetic product comprising the colourant-incorporated silk fibroin is provided.

### CROSS-REFERENCE TO RELATED APPLICATION

This application claims the benefit of priority of Singapore Patent Application No. 10201610640X, filed 20 Dec. 2016, the content of it being hereby incorporated by reference in its entirety for all purposes.

### TECHNICAL FIELD

The present disclosure relates to a method of producing colourant-incorporated silk fibroin. The present disclosure also relates to the colourant-incorporated silk fibroin and cosmetic products including such colourant-incorporated silk fibroin.

### BACKGROUND

Cosmetics have deeply integrated into the daily life of consumers and are used for aesthetics purposes (e.g. skin care). The influence from the use of cosmetics is immensely widespread and substantial, and almost everyone is exposed to cosmetics in one way or another. Currently, however, cosmetics may contain harmful chemicals. Reports have been made that a large portion of the cosmetics available, estimated to be 80%, may contain ingredients which, on prolonged exposure to humans, may lead to adverse health effects such as cancer, reproductive toxicity and organ toxicity. Such alarming reports continued to surface in recent years.

Conventionally, in cosmetics formulations, dyes may be used together with extender powders. These dyes, when come into contact with human skin, may give rise to adverse effects in the long run (e.g. pigmentation).

The extender powders, such as talc, mica, kaolin, and sericite, have been used as a base material [more than 90 weight percent (wt %)] in powder cosmetics to provide spreadability and adhesion onto the skin. However, they are often unsuitable for application to the skin due to being microsized or macrosized. For this reason, the extender powders are often reduced to a sub-micrometer size range, but this leads to absorption of moisture and/or lipids from the skin, thereby causing dehydration and/or de-lipidation of the skin.

Moreover, many ingredients of cosmetics are classifed as industrial chemicals. One example is mica (potassium aluminum silicate) which has been extensively used in conventional cosmetics. According to the USA Centers for Disease Control and Prevention, inhalation of mica causes scarring in lungs and/or lung diseases (e.g. pneumoconiosis). In addition, mica does not degrade over the long term, and this may be harmful to the body as it can then accumulate within the body. There is also the possibility that chemicals in the cosmetics seep into the bloodstream and damage the organs over time.

In summary, growing consumer awareness of the above issues has led to demand for effective cosmetics and/or cosmetic formulations that provide for aesthetical and functional effects in a safe and healthy manner. There is also demand for cosmetics and/or cosmetic formulations which hydrate (or at least does not dehydate) the skin, and maintain the suppleness of the skin, on extended use/application.

There is thus a need to provide for a cosmetic product that resolves and/or ameliorates one or more of the issues mentioned above, at least mitigating the adverse effects of chemicals (e.g. dyes) in cosmetics, which would include mitigating the adverse effects from their prolonged use.

There is also a need to provide for a method that serves as a solution for meeting the demand to design and/or produce safe and high quality cosmetics, wherein the cosmetics provided by the solution at least address the adverse effects arising from the use of chemical (e.g. dyes) as mentioned above.

### SUMMARY

In one aspect, there is provided for a method of producing a colourant-incorporated silk fibroin, comprising:

providing a solution comprising decrystallized silk fibroin with crystalline-forming regions and one or more colourants; and

recrystallizing the decrystallized silk fibroin to change at least a portion of the crystalline-forming regions into recrystallized regions which confine the one or more colourants, thereby obtaining the colourant-incorporated silk fibroin.

In another aspect, there is provided for a colourant-incorporated silk fibroin obtained according to the method as described above, wherein the silk fibroin consists of silk fibroin from Bombyx mori silkworm.

In another aspect, there is provided for a cosmetic product comprising the colourant-incorporated silk fibroin as described above.

### BRIEF DESCRIPTION OF THE DRAWINGS

The drawings are not necessarily to scale, emphasis instead generally being placed upon illustrating the principles of the invention. In the following description, various embodiments of the present disclosure are described with reference to the following drawings, in which:

FIG. 1A is a schematic diagram showing a summarized process of the present method.

FIG. 1B is used to illustrate the difference between raw fibroin and degummed fibroin. In the left image, sericin coats the raw fibroin fibers. The arrow represents a degumming process and the right image shows degummed fibroin (i.e. removed of sericin).

FIG. 2A shows the colourant-incorporated silk fibroins after freeze-drying (left image) and after grinding to obtain fine coloured silk fibroin powder (right image), wherein the colourants used were organic dyes.

FIG. 2B shows the colour-incorporated silk fibroins after freeze-drying (left image) and after grinding to obtain fine coloured silk fibroin powder (right image), wherein the colourants used were inorganic dyes.

FIG. 3 shows the absorbance spectra of colour-incorporated silk fibroin with their respective absorbance peaks of colourants, demonstrating incoporation of colourants in fibroin.

FIG. 4A shows a CIE (International Commission on Illumination) chromaticity graph depicting the different colours of colourants that can be encapsulated in crystalline β-sheets of fibroin. Mixing of different colour organic dyes generates new colours.

FIG. 4B shows examples of cosmetic powders derived from colour-tunable and intensity-tunable colourant-incorporated silk fibroin.

FIG. 5A shows the characteristic peak of crystalline β-sheet for the X-ray diffraction spectrum of various colourant-incorporated fibroin samples, wherein the colourants used were organic dyes.

FIG. 5B shows the characteristic peak of crystalline β-sheet for the X-ray diffraction spectrum of various colourant-incorporated fibroin samples, wherein the colourants used were inorganic dyes.

FIG. 6 shows the metabolic viability of human dermal fibroblasts after incubation with colourant-incorporated silk fibroin of different concentrations up to 1000 μg/mL over 24 hours. The colourant-incorporated silk fibroin exhibited significant biocompatibility.

### DETAILED DESCRIPTION

The following detailed description refers to the accompanying drawings that show, by way of illustration, specific details and embodiments in which the invention may be practised.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The present disclosure relates to a method of incorporating one or more colourants into silk fibroin to develop coloured silk fibroin. The coloured silk fibroin may be used in the formulation of cosmetics. The present disclosure also relates to cosmetics with such colourant-incorporated silk fibroin. In the present disclosure, the term “silk fibroin” is used interchangeably with “fibroin”. The present method is briefly discussed as follows.

Raw silk typically comprises sericin and fibroin. The sericin may be first removed from fibroin. The fibroin may comprise regions that are crystalline. An example of such crystalline regions may be the ordered β-sheets of fibroin. The term “crystalline”, as used herein, refers to an arrangement having perceptible organization, regularity, or orientation of its constituent elements. As an example, a crystalline region of fibroin means that a part of the fibroin protein is arranged to have a form of perceptible organization, regularity, or orientation of its constituent elements, such that there is an order to the structure in that part of fibroin.

The fibroin is then decrystallized. This means the crystalline regions in the fibroin are changed to one that exhibits no readily perceptible organization, regularity, or orientation of its constituent elements.

One or more colourants are then incorporated into the decrystallized fibroin by recrystallization to form the colourant-incorporated silk fibroin. In the context of the present disclosure, the term “recrystallization” refers to changing or restoring regions of fibroin that have been decrystallized, into crystalline regions, such that an orderly arrangement of fibroin proteins exists in those regions. Decrystallized regions that are recrystallized may be called recrystallized regions in the present disclosure. Due to recrystallization, the one or more colourants become entrapped within the matrix of the resultant fibroin. Entrapment of one or more colourants in the fibroin prevents toxicity issues arising from dye pigment leaching, direct contact with skin and absorption through skin.

The silk fibroin may be obtained from silk. The silk may be from Bombyx mori (B. mori) silkworm, where fibroin is in its natural form. Silk fibroin is used as an ingredient for cosmetics and/or its formulation in the present method, as it provides a moisture-balancing effect, a desirable texture for use as a cosmetic ingredient, ultraviolet (UV) protection, and possesses anti-oxidant property, anti-bacterial property etc. As such, B. mori silkworm's silk is advantageously used as a base material in the present method, for incorporation with one or more colourants (e.g. organic dyes and/or inorganic dyes), to develop safe and functional coloured cosmetics. This eliminates the use of extenders (e.g. in their powder form) such as mica, talc, sericite or kaolin as a base material. The fibroin also reduces direct contact of one or more colourants with the skin, thereby preventing adverse effect(s) (e.g. premature ageing, dehydration, absorption into bloodstream) arising from the contact. In contrast to conventional cosmetics with harmful chemicals, prolong use of the present silk based cosmetic may restore healthy, moisture-balanced, smooth and radiant skin.

Besides producing single-coloured cosmetics from colourant(s) approved by the U.S. Food and Drug Administration (FDA), multi-coloured silk based cosmetics can also be obtained by tuning the ratio of different colourants or different colourant-incorporated fibroin, when used in combination. The present method, accordingly, provides for intrinsic colouring via the entrapped colourants in the fibroin, giving rise to a new generation of cosmetics with improved quality, wherein the one or more colourants may be fused in the silk based material.

Having outlined various advantages of the present method and the present colourant-incorporated silk fibroin for cosmetics, definitions of certain terms are first discussed before going into details of the various embodiments.

The word “substantially” does not exclude “completely” e.g. a composition which is “substantially free” from Y may be completely free from Y. Where necessary, the word “substantially” may be omitted from the definition of the invention.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance.

As used herein, the term “and/or” includes any and all combinations of one or more of the associated listed items.

As used herein, the phrase of the form of “at least one of A and B” may include A or B or both A and B. Correspondingly, the phrase of the form of “at least one of A and B and C”, or including further listed items, may include any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

Details of the various embodiments are now described below.

In the present disclosure, there is provided for a method of producing a colourant-incorporated silk fibroin. The method may comprise providing a solution comprising decrystallized silk fibroin with crystalline-forming regions and one or more colourants, and recrystallizing the decrystallized silk fibroin to change at least a portion of the crystalline-forming regions into recrystallized regions which confine the one or more colourants, thereby obtaining the colourant-incorporated silk fibroin.

In the present method, the silk fibroin refers to fibroin that has been degummed. That is to say, the fibroin is separated from or removed of sericin. Accordingly, in the present disclosure, silk fibroin refers to fibroin that does not contain sericin. In this regard, the present method may first involve a step of separating or removing sericin from fibroin.

Subsequently, the present method may involve preparing a solution of decrystallized fibroin. This includes mixing the fibroin with an acidic solution or a metal salt solution to decrystallize the fibroin. The mixing may involve dissolving the fibroin in the acidic solution or metal salt solution. In various embodiments of the present method, providing the solution may therefore comprise or consist of decrystallizing a silk fibroin with an acidic solution or a metal salt solution.

Decrystallizing the fibroin may cause crystalline regions of fibroin to lose their crystalline structure as it weakens the forces (e.g. van der Waals forces, hydrogen bond) that stabilize such crystalline regions. Such crystalline regions may be called crystallites or nanocrystallites in the present disclosure. By destabilizing the crystalline regions through decrystallization, voids may be created for allowing entities, such as solvent molecules, solvent ions, and/or the one or more colourants, to penetrate and/or become interspersed in the matrix of the fibroin, particularly the crystalline regions that have been decrystallized.

The crystalline regions that are subjected to decrystallization may be partially and/or completely restorable of its crystalline structure, i.e. form back the crystalline regions partially and/or completely. Such restorable crystalline regions, which have been decrystallized, may be called crystalline-forming regions. In various embodiments, the β-sheets of fibroin can be decrystallized and subsequently have their crystalline structure restored.

In embodiments where a metal salt solution is used for decrystallization, decrystallizing the silk fibroin may comprise mixing the silk fibroin with the metal salt solution at a weight to volume ratio of 1:3 to 1:8. In some embodiments, the silk fibroin and metal salt solution may be mixed at a weight to volume ratio of 1:4. These ratios avoid the use of too much and too little metal salt solution. In case of the earlier, more exhaustive removal of salt solution from silk fibroin is required for the purification. In case of the latter, inadequate decrystallization of the silk fibroin may occur.

In various embodiments, decrystallizing the silk fibroin may comprise or consist of heating the acidic solution or the metal salt solution at 50° C. to 70° C. for 2 hours to 6 hours in the presence of the silk fibroin. Heating facilitates infiltration of salt solution to decrystallize the silk fibroin more effectively.

In various embodiments of the present method, the one or more colourants may be added after the fibroin is decrystallized. If decrystallization occurs after adding the one or more colourants, the colourant(s) may be incorporated through adsorption and/or simply infiltrate the silk fibroin. In both cases, the colourants are susceptible to being released from the silk fibroin and this poses the risk of direct skin contact with the colourant(s).

In various embodiments, the acidic solution may be selected from the group consisting of acetic acid, formic acid, hydrochloric acid, nitric acid, and sulphuric acid. In various embodiments, the metal salt solution may be selected from the group consisting of calcium chloride, copper nitrate, lithium bromide, lithium thiocyanate, potassium chloride, and sodium chloride. The acid and metal salt ions weaken the electrostatic interactions in the silk fibroin. To elaborate, van der Waals forces are based on electrostatic attaction between temporary dipoles and induced dipoles caused by movement of electron in atoms and/or molecules. Hydrogen bonds result from electrostatic attaction between two polar groups. The addition of acid and/or metal salt ions, which weakens the electrostatic interactions, in turn weakens the van der Waals forces and hydrogen bonds.

In various embodiments, after decrystallization, the acid or metal salt in the solution may be removed by any suitable means. This implies that, in cases where the metal salt exists as ions that form the metal salt solution, it is the ions that are removed. In cases where the acid dissociates into its ions to form the acidic solution, it is the ions that are removed. In some embodiments, providing the solution may further comprise removing the acid or the metal salt from the decrystallized silk fibroin by dialysis. The removal of the acid and/or metal salt is for obtaining a purified solution of decrystallized fibroin. Other means of obtaining a purified solution of decrystallized fibroin include, without being limited to, gel filtration, salting out and buffer exchange. The one or more colourants may be added after such a purification step.

Once a solution comprising the decrystallized fibroin with crystalline-forming regions and the one or more colourants is prepared, the solution may be prepared for recrystallization. In other words, the crystalline-forming regions of the decrystallized fibroin are to be recrystallized. Recrystallizing the crystalline-forming regions may change at least a portion of and/or all of the crystalline-forming regions into recrystallized regions. This means that the crystalline-forming regions can become partially or completely crystalline by recrystallization.

For recrystallization, a stimulant, for example, external energy, may be applied to the solution. In various embodiments, recrystallizing the decrystallized silk fibroin may comprise or consist of subjecting the solution to a stimulant. The stimulant may comprise mechanical energy, sound energy and/or thermal energy. A stimulant in the form of mechanical energy may be, for example, stirring the solution containing decrystallized silk fibroin and the one or more colourants vigorously. A stimulant in the form of sound energy may be, for example, applying ultrasonication to the solution containing decrystallized silk fibroin and the one or more colourants. A stimulant in the form of thermal energy may be, for example, heating the solution containing decrystallized silk fibroin and the one or more colourants.

Apart from applying a stimulant, recrystallizing the decrystallized silk fibroin may comprise or consist of heating the solution at 50° C. to 65° C. for 1 minute to 60 minutes. In some embodiments, recrystallization may be carried out just by heating the solution. In some embodiments, recrystallization may be carried out by applying more than one stimulant, either separately or simultaneously. For example, sound energy in the form of ultrasonication may be first applied, followed by heating. Applying a second stimulant separately, for example, the heating step, may help in complete recrystallization. If more than one stimulant is applied simultaneously, recrystallization may be accelerated.

When recrystallization occurs, the crystalline-forming regions may change or may become arranged into crystalline regions, i.e. recrystallized regions. As a result of such a change, the one or more colourants may be entrapped and/or confined in the recrystallized regions. For example, the one or more colourants may be confined within the ordered. β-sheets. Advantageously, as the one or more colourants are confined within the recrystallized regions, the colourants are prevented from coming into contact with the skin when the silk fibroin is applied thereon, as a cosmetic product. This mitigates and/or eliminates one or more of the adverse effects mentioned above. It is also advantageous in that the recrystallized regions may be used to entrap and/or confine other harmful chemicals used in formulating cosmetics to prevent direct contact with skin, thereby preventing adverse effect(s) that could have arisen from such contact. The one or more colourants not confined in the recrystallized regions are susceptible to leaching from the fibroin.

In the present method, any suitable colourant(s) for cosmetics may be used. In various embodiments, the one or more colourants may be selected from the group consisting of sodium 4-(3-(2,4-di-Me-Ph-azo)-2,4-di-HO-Ph-azo)-benzenesulphonate (D&C brown 1), disodium; 2-[[4-[ethyl-[(3-sulphonatophenyl)methyl]amino]phenyl]-[4-[ethyl-[(3-sulphonatophenyl)methyl]azaniumylidene]cyclohexa-2,5-dien-1-ylidene]methyl]-5-hydroxybenzenesulphonate (FD&C green 3), 4′,5′-dibromofluorescein (D&C orange 5), 2′,4′,5′,7′-tetrabromofluorescein (D&C red 21), fluorescein (D&C yellow 7), monosodium salt of 2-[(9,10-dihydro-4-hydroxy-9,10-dioxo-1-anthracenyl)amino]-5-methyl-benzenesulphonic acid (Ext D&C violet 2), disodium a-[4-(N-ethyl-3-sulphonato benzyl amino)phenyl]-a-[4-(N-ethyl-3-sulphonato benzylamino)cyclohexa-2,5-dienylidene]toluene-2-sulphonate (FD&C blue 1), iron (III) oxide, iron(II) iron(III) oxide, iron (II) oxide, hexaaluminium(3+)ion octasodium trisulphide(2-)hexaorthosilicate (ultramarine blue), and sodium alumino sulpho silicate (ultramarine pink).

In various embodiments, the silk fibroin may consist of silk fibroin from Bombyx mori silkworm.

The present disclosure also provides for a colourant-incorporated silk fibroin obtained according to the method as described above. In various embodiments, the silk fibroin may consist of silk fibroin from Bombyx mori silkworm. This means that the colourant-incorporated silk fibroin contains solely silk fibroin from Bombyx mori silkworm.

Various embodiments of the present method, and advantages associated with various embodiments of the present method, as described above may be applicable to the colourant-incorporated silk fibroin, and vice versa.

In various embodiments, the colourant-incorporated silk fibroin may comprise or consist of one or more colourants incorporated or confined within recrystallized regions of the fibroin. The resultant silk fibroin has the one or more colourants incorporated within the recrystallized regions of fibroin, which differs from cosmetic products where the colourant(s) are physically mixed (without being incorporated into the fibroin structure) or adsorbed on the fibroin.

In various embodiments, the colourant-incorporated silk fibroin may be in the form of powder.

The present disclosure further provides for a cosmetic product comprising the colourant-incorporated silk fibroin as described above. The cosmetic product may comprise or consist of the colourant-incorporated silk fibroin obtained according to the method as described above.

Various embodiments of the present method and the present colourant-incorporated silk fibroin, and advantages associated with various embodiments of the present method and the present colourant-incorporated silk fibroin, as described above may be applicable to the cosmetic product, and vice versa.

While the methods described above are illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

### EXAMPLES

The present disclosure relates to a method of producing colourant-incorporated silk fibroin. The present disclosure also relates to a colourant-incorporated silk fibroin. The present disclosure further relates to a cosmetic product comprising the colourant-incorporated silk fibroin.

Generally, coloured silk fibroin may be obtained by incorporating colourant(s) into the matrix of β-sheet crystallites of silk fibroin, according to the present method. The nano-sized structure (i.e. crystalline regions) of silk fibroin serves as the host for entrapping one or more colourants, thereby preventing leaching of entrapped colourants while providing pearly, luminous healthy-glow effect, when applied to skin, due to the silk fibroin's refractive index changes. As a result, production of biocompatible silk based cosmetics with a broad range of colours may be developed.

The present method, and the present colourant-incorporated silk fibroin, including its uses, are described in the examples below.

### Example 1: Molecular Structure of Silk Fibroin

Raw B. mori silk is a fiber composed of fibroin and sericin. The latter forms a gum coating the fiber. Raw silk is therefore degummed to yield shiny and smooth fibroin. The fibroin, in the form of fibers, is typically made up of crystalline β-sheets (i.e. ordered β-sheet nanocrystallites), which may have strong hydrogen bonding and van der Waals forces between the β-sheets. The fibroin may also have amorphous regions with varying degrees of hydrogen bonding.

At the molecular level, fibroin may be a multi-chain protein composed of heavy chain [350 to 391 kilodalton (kDa)], light chain (26 kDa) and glycoproteins (P25, 30 kDa). The heavy chain, main structural component of silk fibroin, is a biopolymer containing discrete blocks of hydrophobic amino acid domains interspersed with hydrophilic domains. The amino acids of heavy chain may include glycine [about 43 to 46 weight percent (wt %)], non-polar alanine (about 25 to 30 wt %), polar serine (about 12 wt %) and polar tyrosine (about 5 wt %), wherein the wt % is based on the fibroin. The repetitive hydrophobic domains may fold into discrete stacks of β-sheet nanocrystallites while the hydrophilic domains may form amorphous domains. Silk fibroin, as an amphiphilic protein, can be compatibly used with other hydrophilic and/or hydrophobic cosmetic ingredients. As the silk fibroin can be converted to its crystalline state, its hydrolysed state, or mixture of such states, it is useful for different applications over a range of personal care products.

### Example 2a: General Synthesis Procedure of Colourant-Incorporated Silk Fibroin

The present method involves decrystallization and recrystallization of silk fibroin to deliver one or more colourants into recrystallizable domains of fibroin. The recrystallization of fibroin entraps the one or more colourants in a matrix of the recrystallized β-sheet crystallites. This provides flexibility to incorporate a variety of materials other than colourants, such as hydrating agents, anti-oxidants, fragrances etc.

Prior to adding the colourant(s), the crystalline β-sheets are first decrystallized by using an acid and/or a metal ion salt solution. The acid may include strong and/or weak acid, such as but without being limited to, acetic acid, formic acid, hydrochloric acid, nitric acid and sulphuric acid. The metal salt solution may include a solution that comprises a metal salt which can dissociate into its ions. The acid and/or metal salt solution should be able to dissolve fibroin and induce decrystallization of the crystalline β-sheets.

Decrystallization is involved so as to weaken the van der Waals forces and hydrogen bonding which stabilizes the crystalline structure of β-sheets. The weakening and/or disruption of the van der Waals forces and hydrogen bonds between layers of the β-sheets introduces voids for solvent penetration such that water soluble colourant(s) can reach recrystallizable domains. Therefore, an adequately decrystallized fibroin allows for infilration of one or more colourants. In various instances, the entire decrystallized fibroin may be recrystallized. That is to say, when silk fibroin is subjected to a mechanical/sound/thermal stimulant, recrystallization of β-sheets is induced.

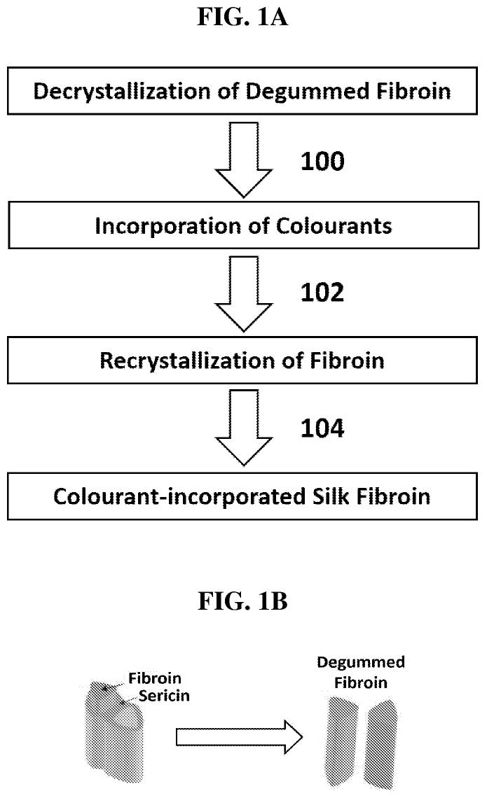

FIG. 1A is schematic diagram showing the summarized process flow of the present method. Generally, in the present method, the degummed fibroin (see FIG. 1B) is first decrystallized. The decrystallized fibroin is then mixed with colourants via 100 for incorporation of colourants. Energy is supplied 102 for the decrystallized fibroin to be recrystallized. The recrystallized fibroin is maintained at a certain energy level (e.g. certain temperature) 104 for complete recrystallization of fibroin, thereby forming the colourant-incorporated silk fibroin.

### Example 2b: Non-Limiting Example of Synthesis of Colourant-Incorporated Silk Fibroin

Decrystallization and recrystallization of silk fibroin for incorporation of one or more colourants, according to embodiments described herein, are described as follow.

The fibroin was decrystallized by dissolving degummed silk fibroin in 9.3 M lithium bromide (LiBr) solution in a weight to volume ratio of 1:4 (1 g silk to 4 mL LiBr). The solution was placed in an oven at 60° C. for 4 hours. Once the silk fibroin was dissolved, silk-LiBr (i.e. fibroin-LiBr) solution was inserted into a hydrated dialysis cassette and dialysed against 1 L of distilled water, which was changed at intervals of 48 hours. After dialysis, the solution was centrifuged at 9000 rpm at 4° C. for 20 minutes to remove impurities and purification was repeated for 3 times. The purified decrystallized fibroin (2 wt/v %) solution was used for incorporation of one or more colourants.

FDA approved colourants were then dissolved in deionized water to prepare 1 mg/mL stock solution (colourant solution). Examples of colourants used include, without being limited to, organic dyes and inorganic dyes. Organic dyes include, without being limited to, D&C brown 1, FD&C green 3, D&C orange 5, D&C red 21, D&C yellow 7, Ext D&C violet 2 and FD&C blue 1. The inorganic dyes include, without being limited to, iron (III) oxide (iron oxide brown), iron(II) iron(III) oxide (iron oxide black), iron oxide green (iron (II) oxide), ultramarine blue and ultramarine pink.

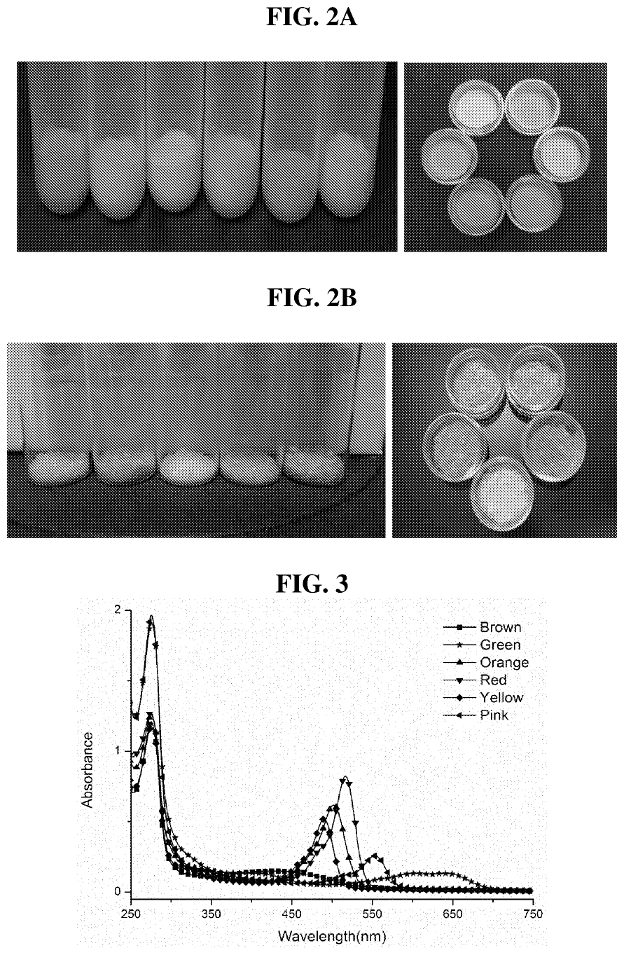

The colourant solution was then mixed with the decrystallized fibroin solution. External energy was applied to distribute local heating and stress in the fibroin solution, thereby promoting β-sheets formation (i.e. recrystallizing the β-sheets). The external energy applied may be any form of energy, for example, mechanical, thermal and/or sound energy. In this process, fibroin becomes recrystallized and the colourant, which has been mixed therein, becomes entrapped (i.e. incorporated) in the matrix of the recrystallized nano-sized β-sheet crystallites. In this example, 0.1 mL of colourant solution was added to 5 mL of the decrystallized fibroin solution, and ultrasonication (a form of sound energy) was applied for 90 seconds at 60% amplitude with an output power of about 12 W to 18 W. Formation of the β-sheets incorporating the one or more colourants was physically induced. The mixture was maintained at 50° C. to 60° C., for 1 minute to an hour, to extend the β-sheets formation (ensure complete recrystallization). The mixture was then frozen overnight at 0° C. to −80° C. It was then freeze-dried for 48 hours and the freeze-dried samples are shown in FIG. 2A (left image). The freeze-dried samples were further grinded to obtain fine powder as depicted in FIG. 2A (right image).

### Example 3a: Characterization of Colour-Incorporated Silk Fibroin

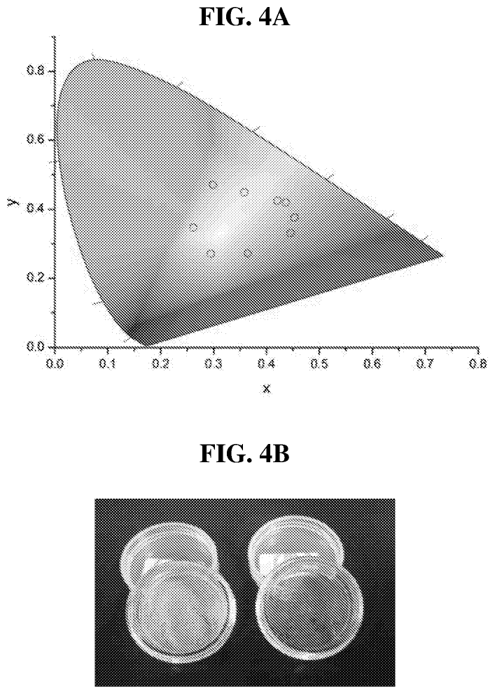

The incorporation of dyes into coloured silk fibroin powder samples was analysed using UV-vis (UV-visible) absorbance spectrometer. Their absorption spectrum substantiated the incorporation of colourant(s) (see FIG. 3). Through mixing of different colourants, new colours could be generated as shown in the chromaticity diagram (FIG. 4A). The chromaticity diagram can be used to predict the colour of silk based cosmetics when more than one colourants are mixed to obtain new colours [e.g. a different colour, different tone (darkness or lightness)].

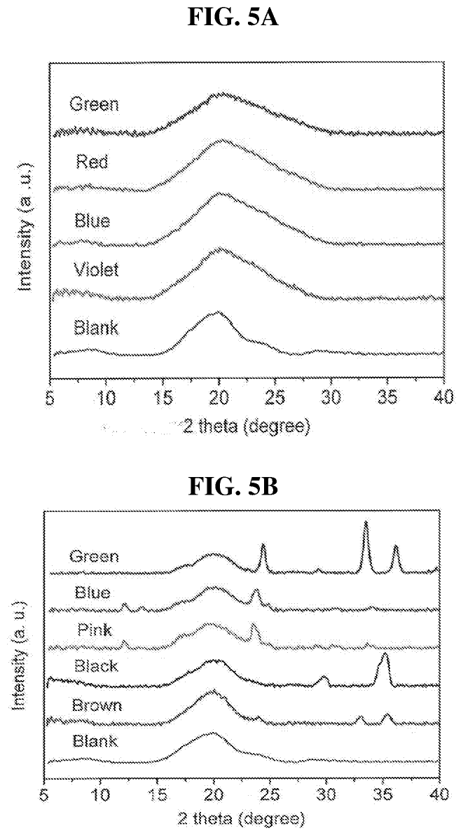

The recrystallized fibroin samples were also analysed using X-ray diffraction (XRD) [radiation wavelength at 1.5418 Å (Cu Kα)] to confirm the formation of crystalline β-sheets. FIG. 5A and FIG. 5B show the representative XRD spectra of the recrystallized fibroin incorporated with colourants. The XRD spectra were fitted into characteristic crystalline peaks of fibroin, and the average crystal size of the crystals were determined from the FWHM (full-width at half-maximum) of the (200) and (120) peaks. Calculations were performed using Scherrer equation:

$$L = \frac{0.9 λ}{FWHM \times cos θ}$$

where L=crystallite size, 0.9 is the Scherrer's constant, λ is the wavelength of incident X-ray (1.5418 Å for Cu Kα radiation), 0 is the peak position, and FWHM is the full-width at half-maximum. All spectra exhibited the characteristic crystalline peak of fibroin at 21°, which corresponds to β-sheet structure with inter β-chain spacing of 4.2 Å. The broadness of the peak reflects the presence of crystal with small size of less than 100 nm. Accordingly, the colourant-incorporated silk fibroin samples attained from recrystallization were calculated to be composed of crystallites ranging from 1.64 nm to 1.72 nm. Based on these sizes, the small crystal size indicates the formation of a colourant-incorporated matrix with many boundaries to enable effective dispersion and trapping of colourants in the fibroin.

### Example 3b: Biocompatibility of Present Colour-Incorporated Silk Fibroin

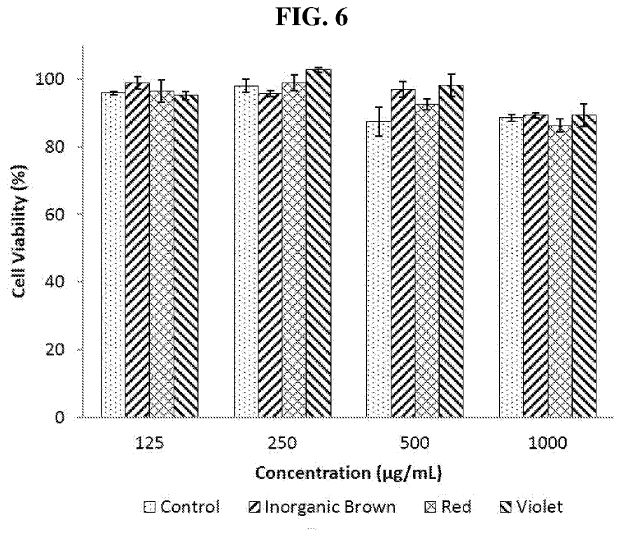

To demonstrate that the present colourant-incorporated fibroin is dermatologically safe for use in cosmetics, biocompatibility of the resultant fibroin products was determined by applying Alamar Blue assay. The samples were incubated with human primary dermal fibroblasts (PCS-201-012 cells, ATCC) for 24 hours to mimic exposure of cosmetics as applied in human makeup. The samples were removed after 24 hours and the cells were rinsed with pre-warmed phosphate-buffered solution before adding Alamar Blue solution for determination of the metabolic activity of cells by measuring fluorescence at 580 nm to 610 nm. The fluorescence readings were directly proportional to the number of living cells. Cell viability was calculated as percentage of living cells comparing to the control cells which had no exposure to the samples. Viability of the cells remained high, averaging more than 90% even when concentration of samples were at 1000 μg/mL over 24 hours exposure (see FIG. 6). The high biocompatibility (i.e. low cytotoxicity) of the coloured silk fibroin ensures their safe applications in cosmetic use.

### Example 4: Comparison of Present Colourant-Incorporated Fibroin and Industry Products

Table 1 below shows a comparison of various parameters between those of present colourant-incorporated fibroin and those of industry products.

TABLE 1
Comparison of Present Colourant-incorporated Fibroin and Industry Products

| | Present Disclosure | Industry Product 1 | Industry Product 2 |
|---|---|---|---|
| | Colourants- | Eye shadow, blush, | Eye shadow, eye |
| | incorporated silk | face powder (Clinique, | liner, blush (Kanebo |
| Parameters | based cosmetics | Maybelline) | Sensai) |
| Colour | Colourants are | Dyes and metal oxide | Colourants mixed |
| | incorporated within | colourants included in | with hydrolysed silk |
| | silk fibroin | these cosmetics may | may give rise to |
| | crystallites and no | directly contact skin and | direct contact with |
| | direct contact with | cause irritation, staining etc. | skin and cause |
| | skin | | irritation, staining |
| | | | etc. |
| Hydration | Naturally | Not an intrinsic property | Although the silk is |
| | moisturizing silk | of the base and colourants. | naturally moisturizing, |
| | fibroin serves as | Requires addition of other | it is only a small |
| | base material | chemicals to impart | fraction of the |
| | | hydration properties | formulation, as the |
| | | | bulk is mica and talc |
| Adhesion | Silk has natural | Require use of extender | Require use of |
| | properties of skin | powder for skin adhesion | extender powder for |
| | affinity and adhesion | and spreadability. | skin adhesion and |
| | | However, they can absorb | spreadability. |
| | | moisture and lipid from | However, they can |
| | | skin to cause dehydration, | absorb moisture and |
| | | de-lipidation and drying of skin | lipid from skin to |
| | | | cause dehydration, |
| | | | de-lipidation and |
| | | | drying of skin |
| Toxicity | Natural silk with no | Talc, mica, dimethicone, | Talc, mica, |
| | toxicity is used as | parabens are some | dimethicone, |
| | the base material to | common ingredients used | parabens are some |
| | replace talc and | in such cosmetics that | common ingredients |
| | mica. The present | raise toxicity issues such | used in such |
| | method encapsulates | as enhanced skin | cosmetics that raise |
| | colourant(s) within | absorption, organ system | toxicity issues such |
| | silk fibroin | toxicity (non-reproductive), | as enhanced skin |
| | crystallites to | endocrine disruption, | absorption, organ |
| | eliminate toxicity. | suspected carcinogens, | system toxicity |
| | Silk based material | allergies, immunotoxicity. | (non-reproductive), |
| | also has anti- | Heavy metals are often | endocrine disruption, |
| | bacterial properties | present which lead to | suspected carcinogens, |
| | and thus remove the | organ system toxicity and | allergies, immunotoxicity. |
| | need for harmful | environmental concerns | |
| | preservatives such | | |
| | as parabens | | |

### Example 5: Commercial and Potential Applications

The above examples demonstrate that the present method can be for preparing silk based materials with controlled combination of colourants to achieve a wide range of desired colours for cosmetic application.

The present method and present colourant-incorporated fibroin demonstrate an incorporation technology that allows colourants to be impregnated in natural silk structure, thus providing protection of skin from direct contact with colourants (i.e. prevents toxicity issues) and also aiding in balancing moisture (i.e. balance hydration) and enhancing radiance of skin. Cosmetics with such colourant-incorporated fibroin, derived from the present method, circumvent the use of harmful unregulated chemicals and/or extenders, such as mica. Such cosmetics also provide aesthetic and functional effects, for example, impart colour, moisture, natural gloss, elasticity and/or luster. The intrinsic colour of such cosmetics, with the colourant-incorporated fibroin, is from the tight arrangement of colourants within the silk fibroin crystallites.

With the above in mind, a thinner layer of such cosmetics can be used to achieve the same aesthetic effects (e.g. natural look and good coverage).

The present method thus provides for a facile and convenient approach to produce coloured and functional cosmetics. Natural, green ingredients are useable in formulating cosmetics based on the present method without sacrificing dermatological safety and personal care.

Apart from the powder form, other forms such as gel, serum, cream etc., can be derived. Additional functionalities, such as anti-ageing, anti-acne etc. can be included.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A method of producing a colourant-incorporated silk fibroin, comprising:
providing a solution comprising decrystallized silk fibroin with crystalline-forming regions and one or more colourants; and
recrystallizing the decrystallized silk fibroin to change at least a portion of the crystalline-forming regions into recrystallized regions which confine the one or more colourants, thereby obtaining the colourant-incorporated silk fibroin.

2. The method according to claim 1, wherein providing the solution comprises decrystallizing a silk fibroin with an acidic solution or a metal salt solution.

3. The method according to claim 2, wherein decrystallizing the silk fibroin comprises mixing the silk fibroin with the metal salt solution at a weight to volume ratio of 1:3 to 1:8.

4. The method according to claim 2 or 3, wherein decrystallizing the silk fibroin comprises heating the acidic solution or the metal salt solution at 50° C. to 70° C. for 2 hours to 6 hours in the presence of the silk fibroin.

5. The method according to any one of claims 2 to 4, wherein the acidic solution is selected from the group consisting of acetic acid, formic acid, hydrochloric acid, nitric acid, and sulphuric acid.

6. The method according to any one of claims 2 to 4, wherein the metal salt solution is selected from the group consisting of calcium chloride, copper nitrate, lithium bromide, lithium thiocyanate, potassium chloride, and sodium chloride.

7. The method according to any one of claims 1 to 6, wherein providing the solution comprises removing an acid or a metal salt from the decrystallized silk fibroin by dialysis.

8. The method according to any one of claims 1 to 7, wherein recrystallizing the decrystallized silk fibroin comprises subjecting the solution to a stimulant.

9. The method according to any one of claims 1 to 8, wherein the stimulant comprises mechanical energy, sound energy and/or thermal energy.

10. The method according to any one of claims 1 to 9, wherein recrystallizing the decrystallized silk fibroin comprises heating the solution at 50° C. to 65° C. for 1 minute to 60 minutes.

11. The method according to any one of claims 1 to 10, wherein the one or more colourants are selected from the group consisting of sodium 4-(3-(2,4-di-Me-Ph-azo)-2,4-di-HO-Ph-azo)-benzenesulphonate, disodium; 2-[[4-[ethyl-[(3-sulphonatophenyl)methyl]amino]phenyl]-[4-[ethyl-[(3-sulphonatophenyl)methyl]azaniumylidene]cyclohexa-2,5-dien-1-ylidene]methyl]-5-hydroxybenzenesulphonate, 4′,5′-dibromofluorescein, 2′,4′,5′,7′-tetrabromofluorescein, fluorescein, monosodium salt of 2-[(9,10-dihydro-4-hydroxy-9,10-dioxo-1-anthracenyl)amino]-5-methyl-benzenesulphonic acid, disodium a-[4-(N-ethyl-3-sulphonato benzyl amino)phenyl]-a-[4-(N-ethyl-3-sulphonato benzylamino)cyclohexa-2,5-dienylidene]toluene-2-sulphonate, iron (III) oxide, iron(II) iron(III) oxide, iron (II) oxide, hexaaluminium(3+)ion octasodium trisulphide(2−)hexaorthosilicate, and sodium alumino sulpho silicate.

12. The method according to any one of claims 2 to 11, wherein the silk fibroin consists of silk fibroin from Bombyx mori silkworm.

13. A colourant-incorporated silk fibroin obtained according to the method of any one of claims 1 to 12, wherein the silk fibroin consists of silk fibroin from Bombyx mori silkworm.

14. The colourant-incorporated silk fibroin according to claim 13, wherein the colourant-incorporated silk fibroin is in the form of powder.

15. A cosmetic product comprising the colourant-incorporated silk fibroin according to claim 13 or 14.
